(19) United States
(12) Patent Application Publication
(10) Pub. No.: US 2011/0066056 A1
(43) Pub. Date: Mar. 17, 2011

(54) EXERCISE AUXILIARY DEVICE

(76) Inventors: Chenghua Huang, Hsi Chih (TW); Yung-Chi Tsai, Hsi Chih (TW); Wen-Hui Chang, Hsi Chih (TW)

(21) Appl. No.: 12/557,628

(22) Filed: Sep. 11, 2009

(51) Int. Cl. A61B 5/04

(52) U.S. Cl. 600/519

(57) ABSTRACT

The exercise auxiliary device comprises a body, a fastener, an ear-clip heart rate sensor, a microprocessor and an indicator. The fastener is mounted on the body for attaching the body to a garment on a user. The ear-clip heart rate sensor is adapted to clip an earlobe of the user, senses blood pulse from the earlobe and accordingly generates a voltage signal. The microprocessor is mounted in the body, electrically connected to the ear-clip heart rate sensor, computes an instantaneous heart rate according to the voltage signal. The indicator is connected to the microprocessor, controlled by the microprocessor to generate a reminding signal. The exercise auxiliary device can be easily worn by attaching the body to a garment of a user with the fastener and clipping an earlobe with the ear-clip heart rate sensor.

### BACKGROUND OF THE INVENTION

1. Field of the Invention

The present invention relates to an exercise auxiliary device, especially to an exercise auxiliary device that monitors heart rate and is easy to wear.

2. Description of the Related Art

For many applications, especially in exercise or fitness training, a person often measures his heart rate to monitor his physical condition. When doing exercise, the person may want to keep his heart rate in a desired range that his body can receive certain benefits from exercise without any adverse effect.

A conventional means to monitor heart-rate during exercising is to wear a chest belt with a heart rate sensor. The user may take off his clothes at first and then wear the chest belt to let the heart rate sensor directly contact with the left side of the chest for precise measurement. However people may feel uncomfortable wearing the chest belt during exercising. For women, it is inconvenient to wear the chest belt in public places. Besides, the chest belt may absorb sweat and thereby cannot be shared with other persons while considering hygiene factors.

Another conventional means is wearing a headset with a heart rate sensor, which also makes people feel uncomfortable during exercising.

To overcome the shortcomings, the present invention provides an exercise auxiliary device in order to mitigate or obviate the aforementioned problems.

### SUMMARY OF THE INVENTION

The main objective of the present invention is to provide an exercise auxiliary device that monitors heart rate and is easy to wear.

The exercise auxiliary device comprises a body, a fastener, an ear-clip heart rate sensor, a microprocessor and an indicator. The fastener is mounted on the body for attaching the body to a garment on a user. The ear-clip heart rate sensor is adapted to clip an earlobe of the user, senses blood pulse from the earlobe and accordingly generates a voltage signal. The microprocessor is mounted in the body, electrically connected to the ear-clip heart rate sensor, computes an instantaneous heart rate according to the voltage signal. The indicator is connected to the microprocessor, controlled by the microprocessor to generate a reminding signal. The exercise auxiliary device is easy to use by attaching the body to a garment of a user with the fastener and clipping an earlobe with the ear-clip heart rate sensor.

Other objectives, advantages and novel features of the invention will become more apparent from the following detailed description when taken in conjunction with the accompanying drawings.

### BRIEF DESCRIPTION OF THE DRAWINGS

FIG. 1 is a block diagram of a preferred embodiment of an exercise auxiliary device in accordance with the present invention;

FIG. 2 is a perspective view of a preferred embodiment of the exercise auxiliary device in accordance with the present invention;

FIG. 3 is an operational view of the exercise auxiliary device; and

FIG. 4 is a graph of a heart rate of a user shown by an indicator of the exercise auxiliary device.

### DETAILED DESCRIPTION OF THE PREFERRED EMBODIMENTS

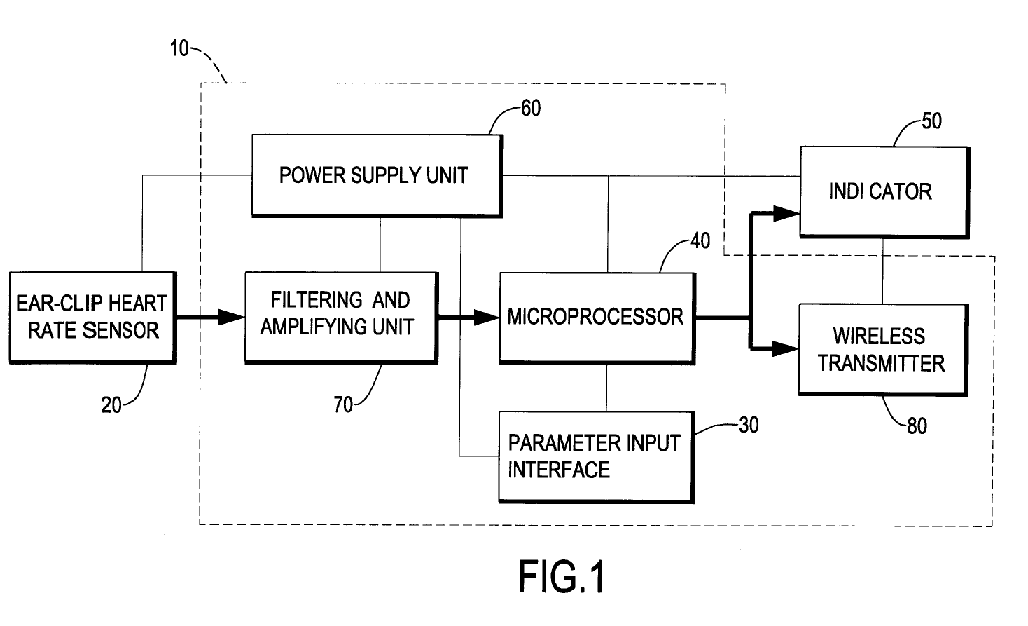

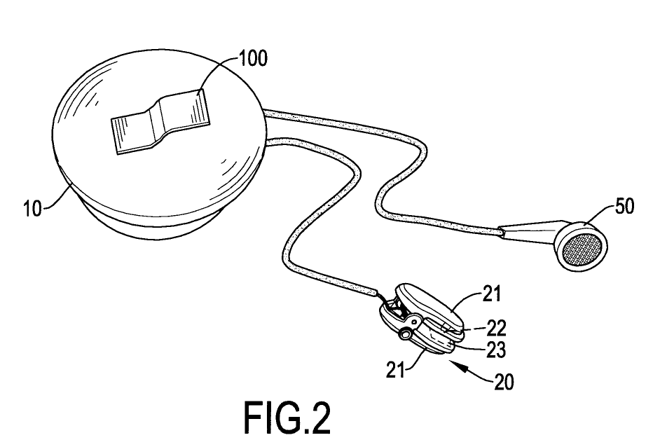

With reference to FIGS. 1, 2 and 3, an exercise auxiliary device in accordance with the present invention comprises a body (10), a fastener (100), an ear-clip heart rate sensor (20), a parameter input interface (30), a microprocessor (40), an indicator (50) and a power supply unit (60).

The fastener (100) is mounted on the body (10) for attaching the body (10) to a garment worn by a user. Preferably the fastener (100) is implemented as a clip for attaching the body (10) to a neckline of the garment.

The ear-clip heart rate sensor (20) is used to clip a user's earlobe to senses a user's blood pulse from his earlobe and accordingly generate a voltage signal. The ear-clip heart rate sensor (20) may have two arms (21), an infrared LED (22) and a photo sensor (23). The arms (21) are opposite to each other. The infrared LED (22) is mounted in one of the arms (21) and emits an infrared light to the earlobe of the user. The photo sensor (23) is mounted in the other arm (21) of the ear-clip heart rate sensor (20) and senses the infrared light through the earlobe of the user to monitor a blood pulse of the user and accordingly generate a voltage signal.

The parameter input interface (30) is mounted on the body (10) for setting physical data of a user, such weight, age or the like and may be implemented as multiple input keys.

The microprocessor (40) is mounted in the body (10), electrically connected to the ear-clip heart rate sensor (20) and the parameter input interface (30), receives the voltage signal and the physical data, computes an instantaneous heart rate according to the voltage signal, computes an optimal heart rate range according to the physical data, compares the instantaneous heart rate with the optimal heart rate range and generates a control signal according to the result from the comparison.

The indicator (50) is electrically connected to the microprocessor (40), receives the control signal and generates a reminding signal accordingly and may be an LED (light-emitting diode) device, a liquid crystal display device, a speaker or a combination of the liquid crystal display device and the speaker, wherein the LED device emits lights as a visual reminding signal, the liquid crystal display device displays images as a visual reminding signal, and the speaker generates sounds as an audible reminding signal.

The power supply unit (60) is mounted in the body (10) and electrically connected to the ear-clip heart rate sensor (20), the parameter input interface (30), the microprocessor (40) and the indicator (50).

The exercise auxiliary device may further comprise a filtering and amplifying unit (70) and a wireless transmitter (80).

The filtering and amplifying unit (70) is electrically connected to the power supply unit (60) and is electrically connected between the ear-clip heart rate sensor (20) and the microprocessor (40) to filter and amplify the voltage signal from the ear-clip heart rate sensor (20).

The wireless transmitter (80) is electrically connected to the microprocessor (40) and wirelessly transmitting the heart rate computed by the microprocessor (40). The wireless transmitter (80) may be implemented as a radio frequency unit transmitting the heart rate by radio frequency signals or an infrared unit transmitting the heart rate by infrared signals.

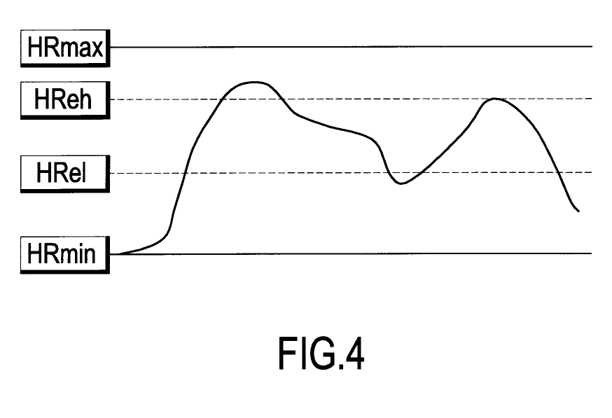

Foregoing optimal heart rate range generated by the microprocessor (40) is a desired range of heart rate one reached during exercise, which enables one to experience the most effective training. Such range varies with a person's physical condition, therefore the optimal heart rate range computed by the microprocessor (40) is based on the physical data of the user. With further reference to FIG. 4, when the indicator (50) is implemented as a liquid crystal display device, the indicator (50) may display a computed optimal heart rate range from HRel to HReh and located between a predetermined minimum heart rate (HRmin) and a maximum heart rate (HRmax). When the heart rate of the exercising user exceeds the range, the indicator (50) may display a conspicuous visual signal as a warning or further cooperate with a speaker to generate an audible warning sound. The user thus knows that the exercise intensity is not beneficial to his/her heart and lungs, then further adjusts the exercise intensity to maintain his/her heart rate in the optimal heart rate range.

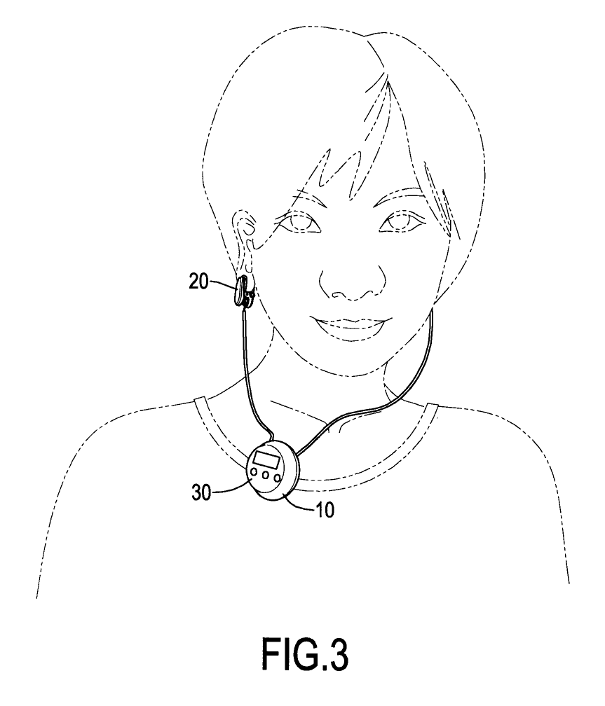

With reference to FIG. 3, the exercise auxiliary device is easy to wear by attaching the body (10) of the exercise auxiliary device to a neckline of a garment of a user with the fastener (not shown) and clip the earlobe of the user with the ear-clip heart rate sensor (20). Therefore the user will not feel uncomfortable when using the exercise auxiliary device during exercising.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. An exercise auxiliary device comprising:
a body;
a fastener mounted on the body for attaching the body to a garment on a user;
an ear-clip heart rate sensor adapted to clip an earlobe of the user, sensing blood pulse from the earlobe and accordingly generating a voltage signal;
a microprocessor mounted in the body, electrically connected to the ear-clip heart rate sensor, receiving the voltage signal, computing an instantaneous heart rate according to the voltage signal; and
an indicator electrically connected to the microprocessor, controlled by the microprocessor to generate a reminding signal.

2. The exercise auxiliary device as claimed in claim 1, wherein the fastener is a clip.

3. The exercise auxiliary device as claimed in claim 1 further has a parameter input interface mounted on the body for setting physical data of a user, wherein the microprocessor is electrically connected to the parameter input interface, receives the physical data computes an optimal heart rate range according to the physical data, compares the instantaneous heart rate with the optimal heart rate range and generates a control signal according to the result from the comparison; and
the indicator connected to the microprocessor receives the control signal and generates a reminding signal accordingly.

4. The exercise auxiliary device as claimed in claim 2 further has
a parameter input interface mounted on the body for setting physical data of a user, wherein the microprocessor is electrically connected to the parameter input interface, receives the physical data computes an optimal heart rate range according to the physical data, compares the instantaneous heart rate with the optimal heart rate range and generates a control signal according to the result from the comparison; and
an indicator electrically connected to the microprocessor, receiving the control signal and generating a reminding signal accordingly.

5. The exercise auxiliary device as claimed in claim 3 further has a power supply unit mounted in the body and electrically connected to the ear-clip heart rate sensor, the parameter input interface, the microprocessor and the indicator.

6. The exercise auxiliary device as claimed in claim 5 further has a filtering and amplifying unit electrically connected between the ear-clip heart rate sensor and the microprocessor, filtering and amplifying the voltage signal from the ear-clip heart rate sensor.

7. The exercise auxiliary device as claimed in claim 6 further has a wireless transmitter electrically connected to the microprocessor and wirelessly transmitting the heart rate.

8. The exercise auxiliary device as claimed in claim 3, wherein the indicator is an LED (light-emitting diode) device that emits lights as a visual reminding signal.

9. The exercise auxiliary device as claimed in claim 3, wherein the indicator is a liquid crystal display device that displays images as a visual reminding signal.

10. The exercise auxiliary device as claimed in claim 3, wherein the indicator is a speaker that generates sounds as an audible reminding signal.

11. The exercise auxiliary device as claimed in claim 3, wherein the indicator includes
a liquid crystal display device displaying images as a visual reminding signal; and
a speaker generating sounds as an audible reminding signal.

12. The exercise auxiliary device as claimed in claim 7, wherein the wireless transmitter is a radio frequency unit transmitting the heart rate by radio frequency signals.

13. The exercise auxiliary device as claimed in claim 7, wherein the transmitter is an infrared unit transmitting the heart rate by infrared signals.
